(19) United States
(12) Patent Application Publication
(10) Pub. No.: US 2008/0187561 A1
(43) Pub. Date: Aug. 7, 2008

(54) Enteric Coated Didanosine Compositions Having A Subcoat

(76) Inventors: Mona DHALIWAL, Saharanpur (IN); Girish Karanth, Shimoga (IN); Romi Barat Singh, Varanasi (IN)

Correspondence Address: Jayadeep R. Deshmukh, Esq.;Ranbaxy Inc., 600 College Road East, Suite 2100, Princeton, NJ, 08540, US

(21) Appl. No.: 11/963,191

(22) Filed: Dec. 21, 2007

(30) Foreign Application Priority Data
Jan. 2, 2007 (IN) 15/DEL/2007

(51) Int. Cl. A61K 9/00; A61K 31/522

(52) U.S. Cl. 424/400; 514/263.23

(57) ABSTRACT

The present invention relates to enteric coated didanosine compositions having a subcoat comprising hydroxypropyl methylcellulose, and process for preparation thereof. The enteric coating ranges from 19-25% w/w of the composition. The compositions are stable and have acceptable acid resistance and release profile.

### FIELD OF INVENTION

The present invention relates to enteric coated didanosine compositions having a subcoat and process for preparation thereof. The compositions are useful against various retroviral disorders, particularly HIV related infections.

### BACKGROUND OF INVENTION

Didanosine or 2′,3′-dideoxyinosine (ddl) is a synthetic purine nucleoside analogue active against the Human Immunodeficiency Virus (HIV). Intracellularly, didanosine is converted by cellular enzymes to the active metabolite, dideoxyadenosine 5′-triphosphate. Dideoxyadenosine 5′-triphosphate inhibits the activity of HIV-1 reverse transcriptase both by competing with the natural substrate, deoxyadenosine 5′-triphosphate, and by its incorporation into viral DNA causing termination of viral DNA chain elongation.

The various didanosine formulations available in the market include chewable/dispersible buffered tablets, buffered powder for oral solution and enteric coated beadlets filled in capsules.

Didanosine is unstable in acidic solutions. For example, at pH<3 and 37° C., 10% of didanosine decomposes to hypoxanthine in less than 2 minutes.

The stability problem associated with acid sensitive compounds is recognized in the prior art, which teaches enteric coated preparations to prepare stable formulations of these compounds. One of the approaches utilized to prevent the incompatability of the acid sensitive compound and the enteric coating material is the use of subcoat between the core and the enteric coating.

The use of a subcoat between the alkaline core and the enteric coating is described in U.S. Pat. No. 5,225,202, wherein the core includes an acid sensitive drug such as didanosine, an additional subcoat layer, which acts as a physical barrier between the core and outer enteric coating layer, and thus prevents interaction of the acid sensitive drug and the acidic enteric coat. This patent teaches the use of buffering agents to minimize drug degradation in the core.

U.S. Patent Application No. 20010051188 teaches a pharmaceutical composition comprising a core in the form of a spheronized beadlet and an enteric coating for said core, said core comprising about 80% to about 100% by weight of an acid labile medicament which is 2′, 3′-dideoxyinosine, a disintegrant and a binder, said composition being devoid of protective coat or subcoat between the core and the enteric coating.

Indian Application No. 979/DEL/2006 discloses an enteric coated didanosine composition. The composition includes a core comprising didanosine, a subcoat comprising polyvinyl alcohol surrounding the core, and an enteric coating surrounding the subcoat.

PCT application WO06/054175 discloses stable didanosine compositions comprising a core containing didanosine, a seal coat on said core and an enteric coating on said seal coat. The core comprises sodium carboxymethyl cellulose as a binder.

It was observed that the presence of a subcoat between the core and the enteric coating hinders the release of didanosine from the core. We have surprisingly found that composition of didanosine with subcoat having comparable release profile to that of didanosine formulations without subcoat can be prepared when the subcoat comprises of hydroxypropyl methylcellulose.

It was further observed that by optimizing the enteric coating percentage in the composition, didanosine compositions having acceptable acid resistance and release profile could be obtained.

Thus, the present invention relates to pharmaceutical compositions of didanosine comprising a core, a subcoat comprising hydroxypropyl methylcellulose and an enteric coating. The core is free of binder or buffering agent. The compositions are stable and have acceptable acid resistance and release profile. Further, the compositions are easy and economical to manufacture.

### SUMMARY OF THE INVENTION

According to one embodiment of the specification there is provided an enteric coated didanosine composition comprising: a) a core comprising didanosine; b) a subcoat surrounding the core, the subcoat comprising hydroxypropyl methylcellulose; and c) an enteric coating surrounding the subcoat; wherein the enteric coating ranges from 19 to 25% w/w of composition, and the core is free of binders or buffering agents.

According to another embodiment of the specification there is provided an enteric coated didanosine composition comprising: a) a core comprising didanosine; b) a subcoat surrounding the core, the subcoat comprising hydroxypropyl methylcellulose; and c) an enteric coating surrounding the subcoat; wherein the enteric coating ranges from 22 to 24% w/w of composition, and the core is free of binders or buffering agents.

According to further embodiment of the specification there is provided a process for the preparation of enteric coated didanosine composition, the process comprising the steps of: a) preparing a core comprising didanosine; b) coating the core obtained in step (a) with a subcoat comprising hydroxypropyl methylcellulose, and (c) coating the product of step (b) with an enteric coating; wherein the enteric coating ranges from 19 to 25% w/w of composition, and the core is free of binders or buffering agents.

### DETAILED DESCRIPTION OF THE INVENTION

The enteric coated didanosine composition comprises: a) a core comprising didanosine; b) a subcoat surrounding the core, the subcoat comprising hydroxypropyl methylcellulose; and c) an enteric coating surrounding the subcoat.

The term “didanosine” as used herein and in the appended claims refers to didanosine or pharmaceutically acceptable salts thereof.

The “core” as used herein refers to any structure that is enclosed or surrounded by a subcoat. The core may be in the form of beadlets or pellets. The core may also contain one or more pharmaceutically acceptable excipients.

The “pharmaceutically acceptable excipients” may be selected from one or more of diluents, disintegrants, lubricants/glidants, solubilizers/wetting agents and mixtures thereof.

Suitable diluents may be selected from one or more of sugars such as dextrose, glucose and lactose; sugar alcohols such as sorbitol, xylitol and mannitol; cellulose derivatives such as powdered cellulose and microcrystalline cellulose; and starches such as corn starch,

Suitable disintegrants may be selected from one or more of sodium starch glycolate, croscarmellose sodium, crospovidone and corn starch.

The lubricant/glidants may be selected from one or more of magnesium stearate, talc, sodium stearyl fumarate, colloidal silicon dioxide and mixtures thereof.

The “core” is free of any binder or buffering agent.

The term “subcoat” as used herein refers to the layer that separates the core from the enteric coating. The subcoat rapidly dissolves upon or after contact with water. The subcoat comprises hydroxypropyl methylcellulose. The subcoat may further contain one or more of plasticizers, flow aids and colorants.

Plasticizers may include one or more of polyethylene glycol (PEG) 400, lecithin, triethylcitrate, glyceryl triacetate, dibutyl sebacate, diethyl phthalate, glycerol and castor oil. For example, the plasticizer may be low molecular weight polyethylene glycols such as PEG 200, PEG 300, PEG 400 and PEG 600.

The flow aid may be selected from one or more of talc, fumed silica, lactose and starch.

The colorant may be selected from one or more of any food approved colors, opacifiers, and dyes. For example, these may be aluminum lakes, iron oxides, titanium dioxide, or natural

The “enteric coating” as used herein refers to the layer that surrounds the subcoat. The enteric coating comprises of enteric polymers, plasticizers, alkalizing agents and, optionally inert excipients. The enteric coating constitutes 19 to 25% w/w of the composition. For example, the enteric coating constitutes 22-24% w/w of the composition.

The enteric polymers may include one or more of cellulose acetate phthalate, hydroxypropylmethyl cellulose phthalate, polyvinyl acetate phthalate, carboxymethylethylcellulose, methacrylic acid methyl esters/methacrylic acid copolymers, such as for example, compounds known under the tradenames of Eudragit NE30D, Eudragit L, Eudragit S, Eudragit L 100 55, Eudragit L30 D 55 by Rohm Pharma and mixtures thereof.

In order to reduce the incompatibility between the acid labile didanosine in the core and the enteric coating, the pH of the enteric coating polymer needs to be raised by using a suitable alkalizing agent.

The term “alkalizing agent” refers to compounds, which are added to the enteric coating to raise the pH below the point where enteric integrity of the polymer could be lost. Thus they decrease the instability problem when in contact with acid labile ingredients. The alkalizing agent may be selected from one or more of sodium hydroxide, potassium hydroxide, calcium hydroxide, magnesium hydroxide and ammonium hydroxide. For example, the alkalizing agent may be sodium hydroxide.

The enteric coating may also contain plasticizers such as triacetin, triethyl citrate, tributyl sebecate, diethyl phthalate, polyethylene glycol; and inert excipients such as talc, titanium dioxide, colloidal silicon dioxide, hydroxypropyl methylcellulose, crospovidone and mixtures thereof.

The core may be in the form of pellets or beadlets, which may be prepared by conventional processes known to a person skilled in the art, such as by extrusion/spheronization or by coating a pharmaceutically acceptable inert core with solution/suspension comprising didanosine. The core is free of any buffering agents or binders.

The process for the preparation of enteric coated didanosine composition comprises the steps of: a) preparing a core comprising didanosine; b) coating the core obtained in step (a) with a subcoat comprising hydroxypropylmethyl cellulose, and (c) coating the product of step (b) with an enteric coating.

According to one embodiment, the enteric coated composition is bioequivalent to the innovator formulation.

The term “innovator formulation” as used herein refers to capsule formulation of didanosine, commercially available in U.S. under the trade name Videx® EC capsules, from Bristol Myers Squibb.

The following non-limiting examples further illustrate the enteric coated didanosine compositions and process of making thereof:

### EXAMPLES 1-2

| | Example 1 | Example 2 |
|---|---|---|
| | Qty (mg per | Qty (mg per |
| Ingredients | Capsule) | Capsule) |
| Core pellets | | |
| Didanosine | 400 | 400 |
| Sodium starch glycolate | 23 | 23 |
| Purified water | q.s. | q.s. |
| Subcoat | | |
| Hydroxy propyl methyl | 5.100 | 5.100 |
| cellulose | | |
| Polyethylene glycol 400 | 1.700 | 1.700 |
| Talc | 1.275 | 1.275 |
| Titanium dioxide | 0.425 | 0.425 |
| Purified water | q.s. | q.s. |
| Enteric Coating | | |
| Methacrylic acid copolymer | 115.50 | 93.37 |
| Diethyl phthalate | 14.91 | 12.05 |
| Talc | 2.60 | 2.10 |
| Sodium hydroxide | 0.44 | 0.36 |
| Purified water | q.s. | q.s. |
| Lubrication | | |
| Talc | 2.5 | 2.5 |
| Colloidal silicon dioxide | 0.55 | 0.55 |
| Total Weight | 568.0 | 542.425 |

### A. Core Pellets

1. Didanosine and Sodium Starch Glycolate were sifted through #18 BSS (850μ) to obtain a mixture.
2. The mixture of step (1) was mixed in a Rapid Mixer Granulator for 10 minutes to obtain a blend.
3. The blend of step (2) was granulated in Rapid mixer granulator using Purified water to obtain a wet mass.
4. The wet mass obtained in step (3) was extruded in the extruder fitted with 1.2 mm screen so as to get extrudates.
5. The extrudates of step (4) were charged to the spheronizer and were operated to get pellets.
6. The pellets of Step (5) were dried in a suitable drier at 60-70° C. till Loss on drying of pellets was not more than (NMT) 1.5% w/w.
7. Fraction of pellets between #12 BSS (1405μ) and #18 BSS (850μ) were taken for sub coating.

### B. Sub Coating

8. Hydroxy propyl methyl cellulose, polyethylene glycol 400, talc & titanium dioxide were dispersed in purified water under mechanical stirring to obtain a coating dispersion.
9. The core pellets of step (7) were coated with the dispersion of step (8) to achieve the desired weight build up.

### C. Enteric Coating

10. Diethyl phthalate was added to purified water under mechanical stirring, followed by methacrylic acid copolymer under mechanical stirring and was stirred for 15 minutes to obtain a dispersion.
11.1% w/v sodium hydroxide solution was added to above dispersion of step (10) to adjust the pH to 5.0±0.1 and was stirred for 15 minutes.
12. Talc was added to dispersion of step (11) and stirred for 5 minutes.
13. The sub coated pellets of didanosine of step (9) were coated with dispersion of step (12) to achieve the desired weight build up to obtain enteric coated pellets.
14. The enteric coated pellets of step (13) were dried at a product temperature of 30-35° C. for 5-10 minutes.
15. The pellets of step (14) were dried in a suitable drier at 35° C.-40° C. to maintain required loss on drying of NMT 2.0% w/w.

### D. Lubrication

16. Fraction of dried enteric coated pellets of step (15) between #12 BSS (1405μ) and #18 BSS (850μ) were taken for lubrication.
17. Talc and Colloidal Silicon Dioxide were passed through # 36 BSS (420μ) on a mechanical vibratory sifter and were mixed with the enteric coated pellets of step (16) in a non-shear blender for 5 minutes.

### E. Capsule Filling

18. Enteric coated pellets from step (17) were filled in hard gelatin capsules.

### COMPARATIVE EXAMPLE 1

### Without Subcoat

| | Qty (mg per |
|---|---|
| Ingredients | Capsule) |
| Core pellets | |
| Didanosine | 400 |
| Sodium starch glycolate | 23 |
| Purified Water | q.s. |
| Enteric coating | |
| Methacrylic acid copolymer | 87.89 |
| Diethyl phthalate | 11.34 |
| Talc | 1.94 |
| Sodium hydroxide | 0.32 |
| Purified water | q.s. |
| Lubrication | |
| Talc | 2.4 |
| Colloidal silicon dioxide | 0.4 |

### Manufacturing Process

### A. Core Pellets

1. Didanosine and Sodium Starch Glycolate were sifted through #18 BSS (850μ) to obtain a mixture.
2. The mixture of step (1) was mixed in a Rapid Mixer Granulator for 10 minutes to obtain a blend.
3. The blend of step (2) was granulated in Rapid mixer granulator using Purified water to obtain a wet mass.
4. The wet mass obtained in step (3) was extruded in the extruder fitted with 1.2 mm screen so as to get extrudates.
5. The extrudates of step (4) were charged to the spheronizer and were operated to get pellets.
6. The pellets of Step (5) were dried in a suitable drier at 60-70° C. till Loss on drying of pellets was NMT 1.5% w/w.
7. Fraction of pellets between #12 BSS (1405μ) and #18 BSS (850μ) were taken for enteric coating.

### B. Enteric Coating

8. Diethyl phthalate was added to purified water under mechanical stirring, followed by methacrylic acid copolymer under mechanical stirring and was stirred for 15 minutes to obtain a dispersion.
9.1% w/v sodium hydroxide solution was added to above dispersion of step (8) and was stirred for 15 minutes to adjust the pH to 5.0±0.1.
10. Talc was added to dispersion of step (9) and stirred for 5 minutes.
11. The pellets of step (7) were coated with dispersion of step (10) to achieve the desired weight build up to obtain enteric coated pellets.
12. The enteric coated pellets of step (11) were dried at a product temperature of 30-35° C. for 5-10 minutes.
13. The pellets of step (12) were dried in a suitable drier at 35° C.-40° C. to maintain required loss on drying of NMT 2.0% w/w.

### C. Lubrication

14. Fraction of dried enteric coated pellets of step (13) between #12 BSS (1405μ) and #18 BSS (850μ) were taken for lubrication.
15. Talc and Colloidal Silicon Dioxide were passed through # 36 BSS (420μ) on a mechanical vibratory sifter and were mixed with the enteric coated pellets of step (14) in a non-shear blender for 5 minutes.

### D. Capsule Filling

16. Enteric coated pellets from step (15) were filled in hard gelatin capsules.

Assay, Acid Resistance & Dissolution Profile of the above examples are given below:

Table 1: Acid resistance results
Acid resistance test of the enteric-coated pellets in 1000 ml 0.1N HCl at 100 rpm for 120 min.

| | Comparative | |
|---|---|---|
| | Example 1 | Example 1 |
| Assay | 98.80% | 98.4% |
| Acid resistance | nil | 2.0% |

### Table 2: Dissolution Profile

Dissolution profile in 1000 ml 0.1N HCl, at 100 rpm for 2 hrs at 37° C.±0.5° C. using USP type I method followed by pH 6.8 phosphate buffer.

| | Percentage drug released (% w/w) | | |
|---|---|---|---|
| | | | Videx EC |
| Time | Comparative | | capsules |
| (in min) | Example 1 | Example 1 | 400 mg |
| 10 min. | 51 | 48 | 50 |
| 20 min | 87 | 82 | 84 |
| 30 min. | 96 | 94 | 93 |
| 45 min. | 98 | 98 | 95 |
| 60 min. | 98 | 98 | 96 |

The above results show that sub coating with hydroxypropylmethyl cellulose does not delay the release of didanosine in pH 6.8 phosphate buffer. The drug release is comparable to that of the composition without subcoat (comparative example 1).

### Table 3: Stability Data

Stability studies of example 1 capsules were conducted for 3 months at 40° C./75% RH. The data shows that unknown impurity is below 0.1% w/w and also the Impurity A (Hypoxanthine) is within the specified limits, at 3M 40° C./75% RH conditions. This shows that the enteric coated composition is stable throughout the stability conditions.

| | | 3 Month |
|---|---|---|
| IMPURITIES | Initial | 40° C./75% RH |
| Impurity A | 0.238 | 0.571 |
| (Hypoxanthine) | | |
| Highest | 0.055 | 0.060 |
| unknown | | |
| Total Unknown | 0.107 | 0.095 |
| Total Related | 0.390 | 0.666 |
| Substances (RS) | | |

### Table 4: Bioequivalence Study Results

Enteric coated Didanosine capsules according to the present invention (T) was compared with Videx® EC capsules, from Bristol Myers Squibb (R) under fasted condition. The results are given in the table below:

| | Cmax | AUC0-₍₀₋ₜ₎ | AUC0-inf |
|---|---|---|---|
| T/R Ratio | 105.42 | 112.70 | 112.47 |
| (CI) % | (97.05-114.50)% | (104.51-121.54)% | (104.71-120.81)% |

As evident from the above data, enteric coated didanosine composition according to the present invention, is bioequivalent to Videx® EC capsules.

We claim:

1. An enteric coated didanosine composition comprising: a) a core comprising didanosine; b) a subcoat surrounding the core, the subcoat comprising hydroxypropyl methylcellulose; and c) an enteric coating surrounding the subcoat, wherein the enteric coating ranges from 19 to 25% w/w of the composition, and the core is free of binders or buffering agents.

2. The composition according to claim 1, wherein the composition comprises one or more pharmaceutically acceptable excipients in either the core or the subcoat or both.

3. The composition according to claim 2, wherein the pharmaceutically acceptable excipients are selected from one or more of diluents, disintegrants, lubricants, plasticizers, flow aids and colorants.

4. The composition according to claim 3, wherein the diluent is selected from one or more of sugars selected from dextrose, glucose and lactose; sugar alcohols selected from sorbitol, xylitol and mannitol; cellulose derivatives selected from powdered cellulose and microcrystalline cellulose; and starches selected from corn starch, pregelatinized starch and maize starch.

5. The composition according to claim 3, wherein the disintegrant is selected from one or more of sodium starch glycolate, croscarmellose sodium, crospovidone and corn starch.

6. The composition according to claim 3, wherein the lubricant is selected from one or more of magnesium stearate, talc, sodium stearyl fumarate and colloidal silicon dioxide.

7. The composition according to claim 3, wherein the plasticizer is selected from one or more of polyethylene glycol 400, lecithin, triethylcitrate, glyceryl triacetate, dibutyl sebacate, diethyl phthalate, glycerol and castor oil.

8. The composition according to claim 3, wherein the flow aid is selected from one or more of talc, fumed silica, lactose and starch.

9. A process for the preparation of enteric coated didanosine composition, the process comprising the steps of: a) preparing a core comprising didanosine; b) coating the core obtained in step (a) with a subcoat comprising hydroxypropyl methylcellulose, and (c) coating the product of step (b) with an enteric coating; wherein the enteric coating ranges from 19 to 25% w/w of the composition, and the core is free of binders or buffering agents.
